(19) United States
(12) Patent Application Publication
(10) Pub. No.: US 2018/0276406 A1
(43) Pub. Date: Sep. 27, 2018

(54) Healthcare Education Assistance Team System

(71) Applicant: Brian Stalker, Van Nuys, CA (US)

(72) Inventor: Brian Stalker, Van Nuys, CA (US)

(21) Appl. No.: 15/934,994

(22) Filed: Mar. 24, 2018

Related U.S. Application Data
(60) Provisional application No. 62/476,097, filed on Mar. 24, 2017.

(51) Int. Cl. G06F 21/62; G16H 15/00; G06F 17/30; G16H 10/60

(52) U.S. Cl. CPC G06F 21/6245; G16H 15/00; G16H 10/60; G06F 17/30589; G06F 17/30554

(57) ABSTRACT

A system and method to provide a rapid response of informational data and analysis involving members of the medical profession comprising remote databases having federal, state and local business data and licensing registration data; one or more than one processor accessing the remote databases, the processor accesses instructions to search and identify: non-qualified providers, licensing gaps, drug enforcement non-compliance, billing and coding violations, mischaracterization of actual services rendered, prohibited self or cross referrals, and other fraudulent or abusive practices; a central database, where data from the remote databases are is automatically accumulated, stored and assembled in response to user queries; and a user interface for entering queries and displaying results.

### CROSS-REFERENCE TO RELATED APPLICATIONS

This Application claims the benefit under 35 U.S.C. § 119(e) of U.S. Provisional Patent Application Ser. No. 62/476,097, filed on 2017 Mar. 24, the contents of which are incorporated herein by reference in their entirety.

### FIELD OF THE INVENTION

The present invention is in the technical field of medical information and more specifically, to a system to provide a rapid response of informational data and analysis involving members of the medical profession.

### BACKGROUND OF THE INVENTION

The medical profession operates in a very fluid environment. There are many new physicians associated with diagnostic, pharmaceutical and durable medical equipment (DME) providers. Disadvantageously, discovering whether or not these individuals or companies are qualified or licensed in a particular area is nearly impossible for a potential client. The information regard this data is constantly changing and updating. Therefore, there is a need for a system to provide a rapid response of informational data and analysis involving members of the medical profession.

### SUMMARY

The present invention overcomes the limitations of the prior are by providing a rapid response system of informational data and analysis involving members of the medical profession. The system comprises: one or more than one remote database comprising business data and licensing registration data from federal, state and local entities. One or more than one processor communicatively coupled to the one or more than one remote database, where the processor accesses instructions stored on a storage to search and identify: non-qualified providers, licensing gaps, drug enforcement non-compliance, billing and coding violations, mischaracterization of actual services rendered, prohibited self or cross referrals, and other fraudulent or abusive practices. A central database operably connected to the processor, where data from the one or more than one remote database is automatically accumulated, stored and assembled in response to a user query. And a user interface communicatively connected to the one or more than one processor, for entering queries and displaying results.

The data is automatically accumulated and assembled from federal and state criminal and civil investigations and can be optionally entered manually into the system. The instructions stored for use in the processor comprise: an analysis of physicians associated with diagnostic, pharmaceutical and DME providers, an analysis based on a user's search criteria, where independent decisions are to be made with the information provided by the system.

The one or more than one remote database comprise information containing federal, state and local business registrations, federal, state and local licensing registrations, and federal, state and local criminal investigation public records and federal, state and local civil investigation public records, collectively referred to as the information. The information is automatically collected by one or more than one central server and stored in one or more than one storage for analysis and reporting. The information is sorted by relation to a particular individual, company or organization, collectively referred to as an entity, for later retrieval and analysis. The information comprises a provider name, addresses, hyperlinks, Taxpayer Identification Number (TIN), Employer Identification Number (EIN), Qualified medical evaluator (QME), associated providers linked to physician or address, Federal or state licensing information, and National Provider Identifiers (NPI) registrations. A portion of the information can be searched by the user, based upon the user's access level, and the related information will be analyzed and generated in a report that is sent to the user. There are currently three levels of access currently contemplated: a tier one access for a simple name search, a tier two access for a detailed search, or a tier three access for a complex search for names, TIN, business associations and address link analysis. The user can retrieve the information about an entity depending upon an access level granted to the user. The instructions operable on the processor for presenting the user interface comprises options for selecting one or more than one search and analysis criteria that is limited by the access level granted to the user.

There is also provided a method for a rapid response of informational data and analysis involving members of the medical profession. The method comprises the steps where first a user logs onto the system. Then, the user's access level is determined. Next, at least one search criteria is input by the user. Then, the search criteria is analyzed by the system for commonalities and other links of potentially fraudulent activity. Next, a report is generated based upon the search criteria. Finally, the user can request additional support to enhance the report generated from the search criteria.

### BRIEF DESCRIPTION OF THE DRAWINGS

These and other features, aspects and advantages of the present invention will become better understood with regard to the following description, appended claims, and accompanying figures where:

FIG. 1 is a diagram of a system to provide a rapid response of informational data and analysis involving members of the medical profession according to one embodiment;

FIG. 2 is a flowchart diagram of the system of FIG. 1;

FIG. 3 is a screenshot of a home page of the system of FIG. 1;

FIG. 4 is a screenshot of utilities available on the system of FIG. 1;

FIG. 5 is a screenshot of a search history of the system of FIG. 1;

FIG. 6 is a screenshot of a command log of the system of FIG. 1;

FIG. 7 is a screenshot of a capture filter of the system of FIG. 1;

FIG. 8 is a screenshot of a tier 1 report generated by the system of FIG. 1; and

FIG. 9 is a screenshot of a tier 2 report generated by the system of FIG. 1.

### DETAILED DESCRIPTION OF THE INVENTION

The present invention overcomes the limitations of the prior art by providing a system to provide a rapid response of informational data and analysis involving members of the medical profession. The system comprises a database that contains business and licensing registrations from many federal, state and local entities. Data is automatically accumulated and assembled from federal and state criminal and civil investigations. Optionally, the data can be manually entered into the system. The system provides a link analysis of physicians associated with diagnostic, pharmaceutical and DME providers. The system comprises a multifaceted proprietary database for cataloging and supplying accurate information to support anti-fraud initiatives. The system tracks provider qualifications, uncovers counterfeit identities, and links potential financial interest relationships of other providers creating a causal flow diagram. The system can be used for provider vetting to claim strategy defense, to capturing and preventing abusive and fraudulent medical practices. The system does not provide any opinions, recommendations, conclusions or an “action plan” regarding the information provided to subscribers, only a link to an analysis based on a user's search criteria. The system allows independent decisions to be made with the information provided by the system.

All dimensions specified in this disclosure are by way of example only and are not intended to be limiting. Further, the proportions shown in these Figures are not necessarily to scale. As will be understood by those with skill in the art with reference to this disclosure, the actual dimensions and proportions of any system, any device or part of a system or device disclosed in this disclosure will be determined by its intended use.

Methods and devices that implement the embodiments of the various features of the invention will now be described with reference to the drawings. The drawings and the associated descriptions are provided to illustrate embodiments of the invention and not to limit the scope of the invention. Reference in the specification to “one embodiment” or “an embodiment” is intended to indicate that a particular feature, structure, or characteristic described in connection with the embodiment is included in at least an embodiment of the invention. The appearances of the phrase “in one embodiment” or “an embodiment” in various places in the specification are not necessarily all referring to the same embodiment.

Throughout the drawings, reference numbers are re-used to indicate correspondence between referenced elements. In addition, the first digit of each reference number indicates the figure where the element first appears.

As used in this disclosure, except where the context requires otherwise, the term “comprise” and variations of the term, such as “comprising”, “comprises” and “comprised” are not intended to exclude other additives, components, integers or steps.

In the following description, specific details are given to provide a thorough understanding of the embodiments. However, it will be understood by one of ordinary skill in the art that the embodiments may be practiced without these specific details. Well-known circuits, structures and techniques may not be shown in detail in order not to obscure the embodiments. For example, circuits may be shown in block diagrams in order not to obscure the embodiments in unnecessary detail.

Also, it is noted that the embodiments may be described as a process that is depicted as a flowchart, a flow diagram, a structure diagram, or a block diagram. The flowcharts and block diagrams in the figures can illustrate the architecture, functionality, and operation of possible implementations of systems, methods and computer programs according to various embodiments disclosed. In this regard, each block in the flowchart or block diagrams can represent a module, segment, or portion of code, that can comprise one or more executable instructions for implementing the specified logical function(s). It should also be noted that, in some alternative implementations, the functions noted in the blocks may occur out of the order noted in the figures. Although a flowchart may describe the operations as a sequential process, many of the operations can be performed in parallel or concurrently. In addition, the order of the operations may be rearranged. A process is terminated when its operations are completed. A process may correspond to a method, a function, a procedure, a subroutine, a subprogram, etc. When a process corresponds to a function, its termination corresponds to a return of the function to the calling function or the main function. Additionally, each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

Moreover, a storage may represent one or more devices for storing data, including read-only memory (ROM), random access memory (RAM), magnetic disk storage mediums, optical storage mediums, flash memory devices and/or other non-transitory machine readable mediums for storing information. The term “machine readable medium” includes, but is not limited to portable or fixed storage devices, optical storage devices, wireless channels and various other non-transitory mediums capable of storing, comprising, containing, executing or carrying instruction(s) and/or data.

Furthermore, embodiments may be implemented by hardware, software, firmware, middleware, microcode, or a combination thereof. When implemented in software, firmware, middleware or microcode, the program code or code segments to perform the necessary tasks may be stored in a machine-readable medium such as a storage medium or other storage(s). One or more than one processor may perform the necessary tasks in series, distributed, concurrently or in parallel. A code segment may represent a procedure, a function, a subprogram, a program, a routine, a subroutine, a module, a software package, a class, or a combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted through a suitable means including memory sharing, message passing, token passing, network transmission, etc. and are also referred to as an interface, where the interface is the point of interaction with software, or computer hardware, or with peripheral devices.

Various embodiments provide a system to provide a rapid response of informational data and analysis involving members of the medical profession. One embodiment of the present invention provides a system to provide a rapid response of informational data and analysis involving members of the medical profession. In another embodiment, there is provided a method for using the system. The system and method will now be disclosed in detail.

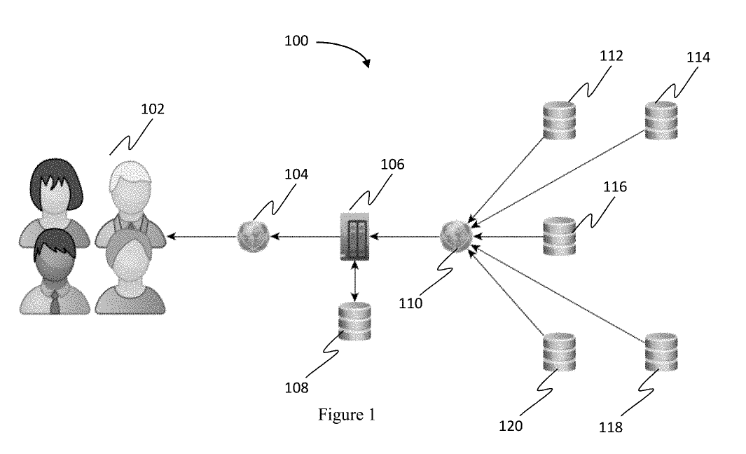

Referring now to FIG. 1, there is shown a diagram of a system 100 to provide a rapid response of informational data and analysis involving members of the medical profession according to one embodiment. The system 100 comprises one or more than one remote databases 1122, 114, 116, 118, 120 and other informational sources that contain business and licensing registrations from many federal, state and local entities and federal and state criminal and civil investigation public records, collectively referred to as the information 110. The information 110 is automatically collected by one or more than one central server 106 and stored in one or more than one storage or one or more than one database 108. The information 110 in the one or more than one storage 108 is then sorted and analyzed by relation to a particular individual, company or organization, collectively referred to as an entity, for later retrieval. A user 104 can log onto the system 104 and, depending upon the access level granted, retrieve information 110 about an entity. The information 110 comprises a provider name, addresses, links, Taxpayer Identification Number (TIN), Employer Identification Number (EIN), Qualified medical evaluator (QME), associated providers linked to physician or address, Federal or state licensing information, and National Provider Identifiers (NPI) registrations. One or more portion of the information 110 can be searched by the user 102 and the related information will be generated in a report that is sent to the user. Currently, it is envisioned that there three levels of searches possible. A simple name search (Level 1) to complex names, TIN, business associations and address link analysis (Level 3). As will be understood by those will skill in the art with reference to this disclosure, there are other possible levels of search possible. The exemplar above is not meant to be limiting.

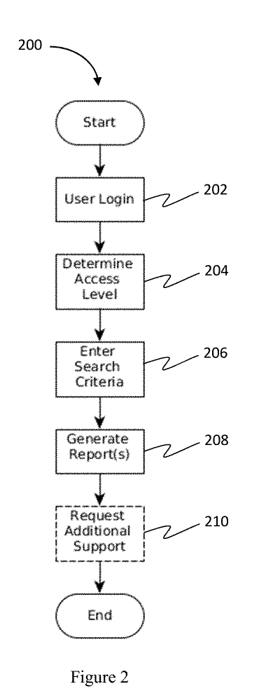

Referring now to FIG. 2, there is shown a flowchart diagram of some steps of a method to implement the system of FIG. 1. What is shown is a computer implemented method for a system to provide a rapid response of informational data and analysis involving members of the medical profession. First, a user logs 202 onto the system. Then, the user's access level is determined 204. Next, a search criterion is 206 input by the user. Next, a report and analysis is generated 208 based upon the search criteria. Optionally, the user can request additional support 210 to enhance the report generated from the search criteria. The system collates and analyzes:

Non-qualified providers

Licensing gaps

Drug Enforcement non-compliance

Billing and coding violations

Mischaracterization of actual services rendered

Prohibited self or cross referrals

Other fraudulent or abusive practices

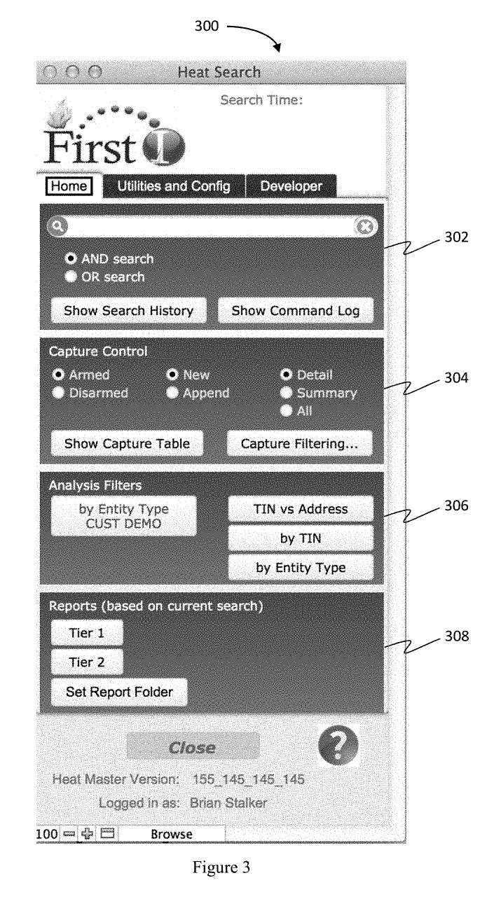

Referring now to FIG. 3, there is shown a screenshot of a home page of the system of FIG. 1. Once the user has been granted access to the system, they are presented with the home page for entering the search criteria. The current home page displays a level 3 access. Other levels of access would either have those areas where access has not been granted greyed out, or optionally, not displayed to the user.

The user enters a name or other information into a search bar. A Boolean logic filter can be added to search for all of the words in the search bar (AND) or any of the information in the search bar (OR). This allows the user to create a broad search and narrow the information parameters down in another search.

The capture control section provides the user options for how the generated report will be displayed. The capture control can also append previously saved searches with a newly entered search. Also, the capture control area comprises additionally filters that can be selected by the user so that the report only comprises the information deemed relevant to the user.

An analysis filter section allows the user to select particular filters to enhance the generated report. The user can select filters that provide analysis of entity type, TIN versus Address, or TIN. The analysis can provide a report showing that the TIN and the address for a give entity match which can indicate that the entity is legitimate.

A report section provides options for the user to display a Tier 1 or Tier 2 report and optionally to save the report on the system. Reports will be generated through an online secured network, or through a hard copy report printed and sent to the user. Optionally, the user can request additional support for the analysis and further review or investigation of the information by qualified personnel.

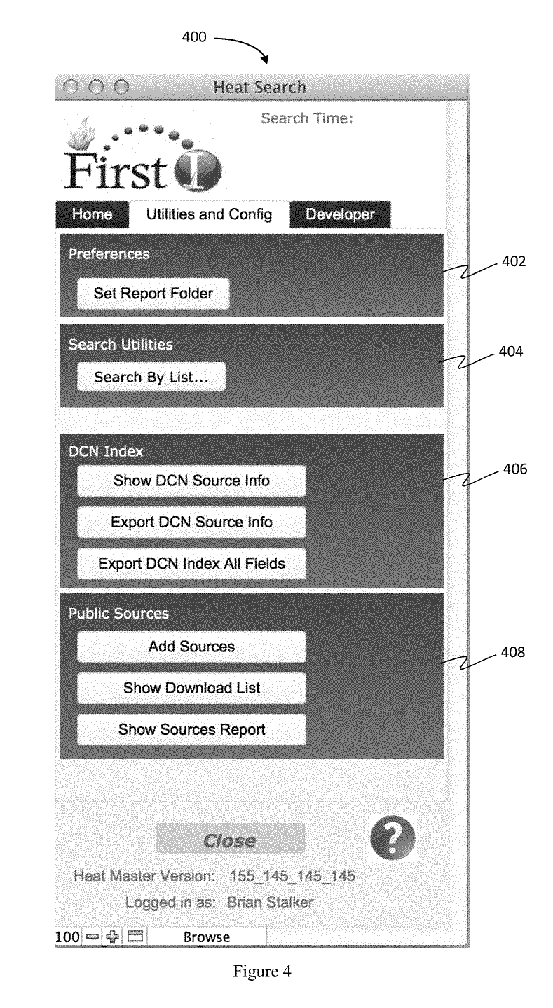

Referring now to FIG. 4, there is shown a screenshot of utilities available on the system of FIG. 1. The utilities available to the user provide customization of the information that is searched and where the reports will be stored for the user. The utilities allow the user to customize the information source to prevent non-relevant data sources from polluting the results and the report. For example, if only doctors were being searched by the user, selecting the DCN index would eliminate any unwanted TIN information from being searched.

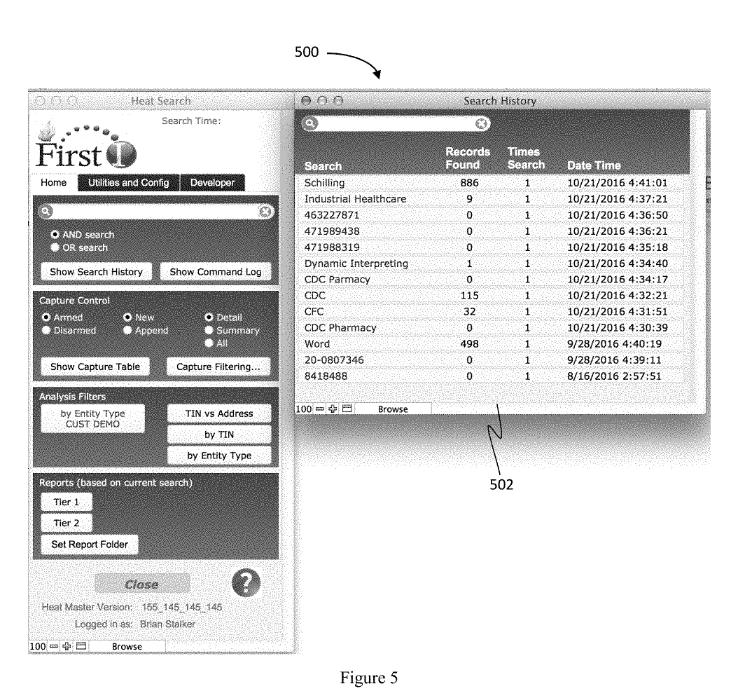

Referring now to FIG. 5, there is shown a screenshot of a search history of the system of FIG. 1. As can be seen, one or more than one saved search conducted by the user is saved in the system in the storage. The one or more than one saved search can then be retrieved from the storage and used to generate a new report using the same criteria, or combining the one or more than one saved search with each other or a new search criterion. This provides the user with a means of re-running reports over a period of time to check for any changes in the information.

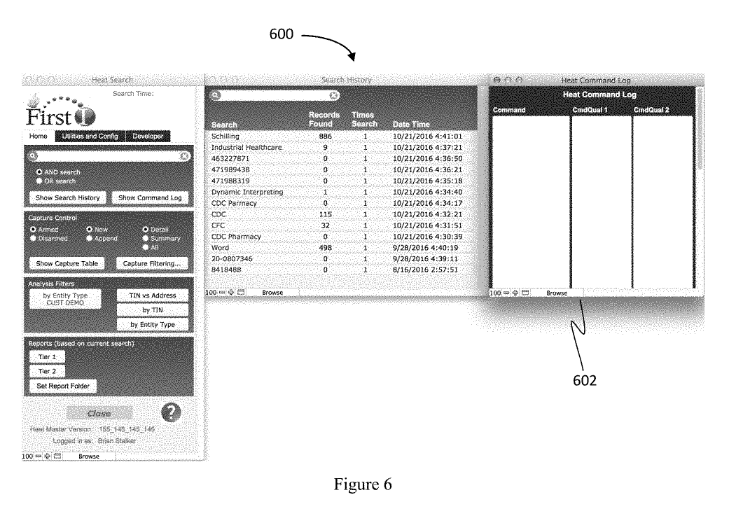

Referring now to FIG. 6, there is shown a screenshot of a command log of the system of FIG. 1. The command log displays the search criteria of the one or more than one saved search so that the user can update the search criteria or verify the search criteria.

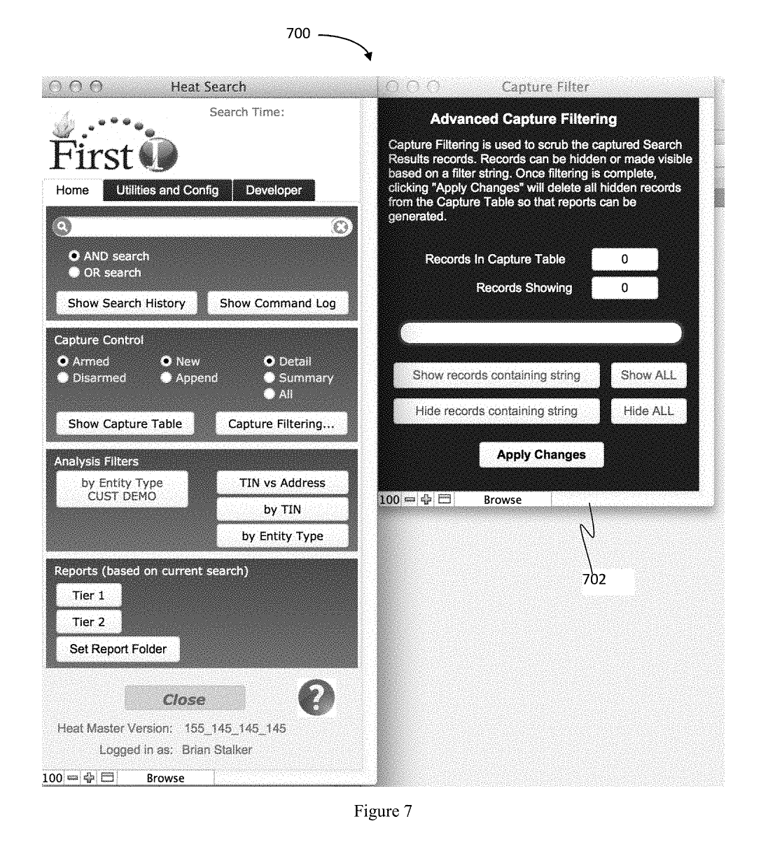

Referring now to FIG. 7, there is shown a screenshot of a capture filter of the system of FIG. 1. As can be seen the capture filtering provide a means for the user to refine or scrub the information retrieved from an initial search. The capture filtering can show more refined results or hide unwanted results thereby reporting only relevant information to the user.

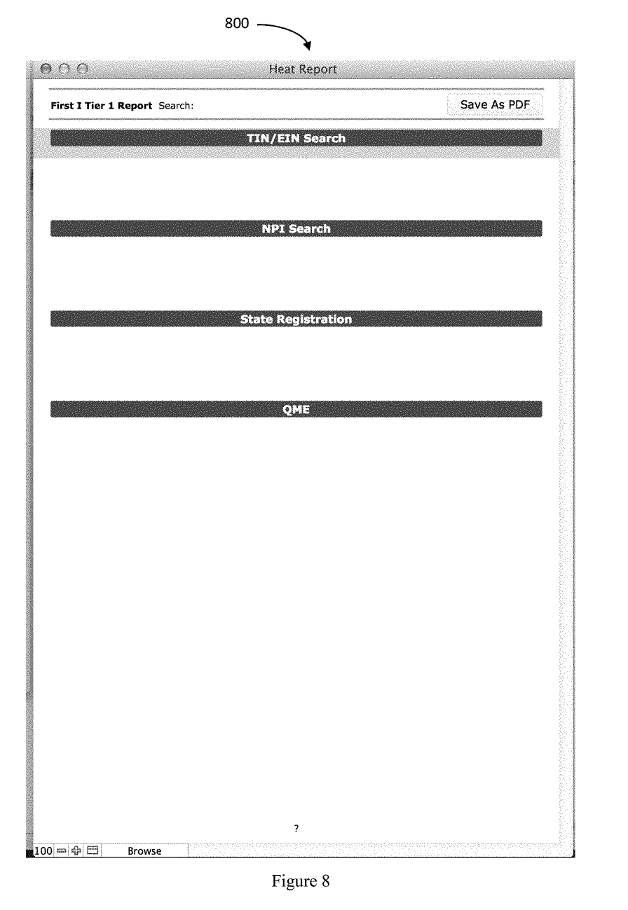

Referring now to FIG. 8, there is shown a screenshot of a tier 1 report generated by the system of FIG. 1. As can be seen the tier 1 report displays TIN/EIN information, NPI search results, State registrations and QME.

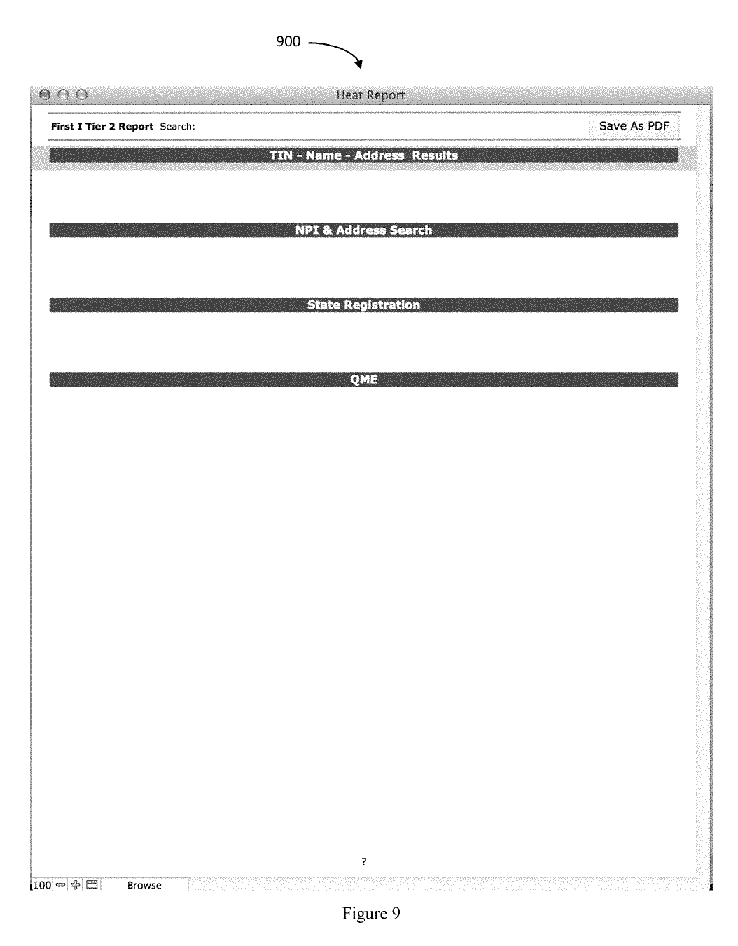

Referring now to FIG. 9, there is shown a screenshot of a tier 2 report generated by the system of FIG. 1. In addition to the information displayed in the tier 1 report, the tier 2 report also displays the addresses associated with the TIN and NPI. This allows the user to confirm information given to them corresponds to official records.

What has been described is a new and improved system for a system to provide a rapid response of informational data and analysis involving members of the medical profession, overcoming the limitations and disadvantages inherent in the related art.

Although the present invention has been described with a degree of particularity, it is understood that the present disclosure has been made by way of example and that other versions are possible. As various changes could be made in the above description without departing from the scope of the invention, it is intended that all matter contained in the above description or shown in the accompanying drawings shall be illustrative and not used in a limiting sense. The spirit and scope of the appended claims should not be limited to the description of the preferred versions contained in this disclosure.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means” for performing a specified function or “step” for performing a specified function should not be interpreted as a “means” or “step” clause as specified in 35 U.S.C. § 112.

What is claimed is:

1. A system to provide a rapid response of informational data and analysis involving members of the medical profession, the system comprising:
a) one or more than one remote database comprising business data and licensing registration data from federal, state and local entities;
b) one or more than one processor communicatively coupled to the one or more than one remote database, where the processor accesses instructions stored on a storage to search and identify: non-qualified providers, licensing gaps, drug enforcement non-compliance, billing and coding violations, mischaracterization of actual services rendered, prohibited self or cross referrals, and other fraudulent or abusive practices;
c) a central database operably connected to the processor, where data from the one or more than one remote database is automatically accumulated, stored and assembled in response to a user query; and
d) a user interface communicatively connected to the one or more than one processor, for entering queries and displaying results.

2. The data of claim 1, where the data is automatically accumulated and assembled from federal and state criminal and civil investigations.

3. The data of claim 1, where the data is optionally entered manually into the system.

4. The system of claim 1, where the instructions stored for use in the processor comprise:
a) instructions for an analysis of physicians associated with diagnostic, pharmaceutical and DME providers; and
b) instructions for an analysis based on a user's search criteria, where independent decisions are to be made with the information provided by the system.

5. The system of claim 1, where the one or more than one remote database comprise information containing federal, state and local business registrations, federal, state and local licensing registrations, and federal, state and local criminal investigation public records and federal, state and local civil investigation public records, collectively the information.

6. The system of claim 5, where the information is automatically collected by one or more than one central server and stored in one or more than one storage for analysis and reporting.

7. The system of claim 6, where the information in the one or more than one storage is sorted by relation to a particular individual, company or organization, collectively referred to as an entity, for later retrieval and analysis.

8. The system of claim 7, where the information comprises a provider name, addresses, hyperlinks, Taxpayer Identification Number (TIN), Employer Identification Number (EIN), Qualified medical evaluator (QME), associated providers linked to physician or address, Federal or state licensing information, and National Provider Identifiers (NPI) registrations.

9. The system of claim 8, where a portion of the information can be searched by the user and the related information will be analyzed and generated in a report that is sent to the user.

10. The system of claim 9 where a user access level is selected from the group consisting of, a tier one access for a simple name search, a tier two access for a detailed search, or a tier three access for a complex search for names, TIN, business associations and address link analysis.

11. The system of claim 8, where the user can retrieve the information about an entity depending upon an access level granted to the user.

12. The system of claim 1, further comprising instructions operable on the processor for presenting the user interface.

13. The system of 12, where the user interface comprises options for selecting one or more than one search and analysis criteria.

14. The system of 13, where the search and analysis criteria is limited by an access level granted to the user.

15. A method to provide a rapid response of informational data and analysis involving members of the medical profession, the steps of the method comprising:
a) logging onto the system by a user;
b) determining the user's access level;
c) inputting at least one search criterion by the user;
d) analyzing the search criteria by the system for commonalities and other links of potentially fraudulent activity;
d) generating a report based upon the search criteria and analysis; and
e) requesting additional support to enhance the report generated from the search criteria by the user.
